(19) United States
(12) Patent Application Publication
(10) Pub. No.: US 2024/0408563 A1
(43) Pub. Date: Dec. 12, 2024

(54) SYSTEMS AND METHODS TO ACCELERATE GEL-SOL TRANSITION FOR THERMORESPONSIVE HYDROGELS

(71) Applicant: University of Southern California, Los Angeles, CA (US)

(72) Inventors: Mark Thompson, Los Angeles, CA (US); Mahsa Rezaiyan, Los Angeles, CA (US); Darius Shariaty, Los Angeles, CA (US); Frances Yau, Los Angeles, CA (US)

(21) Appl. No.: 18/402,685

(22) Filed: Jan. 2, 2024

Related U.S. Application Data
(60) Provisional application No. 63/449,944, filed on Mar. 3, 2023.

(51) Int. Cl. B01J 13/00; A61K 47/32

(52) U.S. Cl. CPC B01J 13/0021; A61K 47/32; B01J 13/0065; B01J 13/0069

(57) ABSTRACT

In some examples, systems and methods are disclosed for enhancing a gel to sol transition of a polymer that includes disposing the polymer on a host material, exposing the polymer and the host material to an exposure temperature that causes the polymer to form a gel, and cooling the polymer and the host material to a cooling temperature that causes the gel to transition to a sol.

### CROSS REFERENCE TO RELATED APPLICATIONS

This application claims priority to U.S. Provisional Appl. No. 63/449,944, filed on Mar. 3, 2023, which is herein incorporated by this reference in its entirety.

### STATEMENT AS TO FEDERALLY SPONSORED RESEARCH

This invention was made with government support under W81XWH-16-C-0086, and W81XWH-21-9-0003 awarded by the Medical Research and Development Command. The government has certain rights in this invention.

### TECHNICAL FIELD

Disclosed herein are systems, structures, and/or methods for enhanced gel-sol transition for hydrogels.

### BACKGROUND

Millions of eye injuries occur each year many of which result in irreversible visual impairment. These injuries can have a severe impact on the life of a patient and invoke an urgent need for delivery of the best outcomes. While ocular trauma is common, patient outcomes can depend on the immediacy of treatment. Studies have shown that treatments at or closest to the time of injury have the best outcomes when dealing with severe ocular trauma. As a result, there is strong demand for a straightforward technology to occlude open globe injury that can be applied by far-forward medical personnel.

Accordingly, a field-ready solution to resolve such injuries that also prevents the deleterious effects of current technology is needed. This disclosure resolves these and other issues of the art.

### SUMMARY

The subject of this disclosure includes a temperature-responsive hydrogel that has enhanced gel to sol transition.

In some examples, a method is disclosed for enhancing a gel to sol transition of a polymer. The method includes disposing the polymer on a host material, exposing the polymer and the host material to an exposure temperature that causes the polymer to form a gel, and cooling the polymer and the host material to a cooling temperature that causes the gel to transition to a sol.

In some aspects, the polymer is a biocompatible thermoresponsive sealant.

In some aspects, the method includes upon cooling the polymer and the host material to the cooling temperature for a cooling duration of time, forming a homogenous temperature-responsive hydrogel with one or more particles of the polymer stored within pores of the host material, the homogenous temperature-responsive hydrogel including a storage and loss moduli that increases with temperature due to enhanced interpolymer interaction.

In some aspects, the cooling duration of time is between about 5 and 15 minutes.

In some aspects, the cooling duration of time is approximately 10 minutes.

In some aspects, the step of exposing the polymer and the host material to the exposure temperature is for an exposure duration of time between about 1 hour and 24 hours.

In some aspects, the step of exposing the polymer and the host material to the exposure temperature is for an exposure duration of time of at least 1 hour.

In some aspects, the step of exposing the polymer and the host material to the exposure temperature is for an exposure duration of time of at least 24 hours.

In some aspects, the exposure temperature is from about 25 to 75° C.

In some aspects, the exposure temperature is approximately 50° C.

In some aspects, the cooling temperature is less than about 25° C.

In some aspects, the cooling temperature is from about −40 to 20° C.

In some aspects, the cooling temperature is approximately 0° C.

In some aspects, the polymer is poly(N-isopropylacrylamide-co-butyl acrylate) (pNIPAM).

In some aspects, the polymer is poly(N-isopropylacrylamide-co-N-tert-butylacrylamide) and poly(N-isopropylacrylamide), poly(N,N-diethyl acrylamide), and poly(methyl vinyl ether), poly(N-vinyl caprolactam), poly(ethylene glycol), poly(propylene glycol), poly(vinylalcohol), poly(N-isopropylacrylamide), poly(methyl vinyl ether), poly(N-vinyl caprolactam), copolymer of poly(ethylene oxide) and poly(propylene oxide) s, and elastin-like oligo- and polypeptides.

In some aspects, a molecular weight of the polymer is at least one of Mn=64 kg mol 1, Mn=41 kg mol 1, Mn=35 kg mol 1, Mn=24 kg mol 1, and Mn=7 kg mol 1.

In some aspects, the host material includes a foam, such as an open-cell foam and a closed-cell foam.

In some aspects, the open-cell foam is selected from the group consisting of polyurethane (PUR), polyimide (PIM), hydrophilic polyurethane (PUR), Aquazone® (AQZ), and neoprene (NEO).

In some aspects, a sol recovery rate resulting from the step of cooling is from about 74 to 99 percent.

In some aspects, the host material includes polyurethane (PUR) foam, and wherein a sol recovery rate resulting from the step of cooling is approximately 100%.

In some aspects, a sol recovery rate resulting from the step of cooling is improved by a factor of about 8-10 relative to the polymer alone without the host material.

In some aspects, pores of the host material range between about 0.2-1.1 mm.

In some aspects, a method is disclosed for reversibly sealing tissue damage. The method includes applying a temperature-responsive hydrogel to a tear or perforation in a tissue of a subject in an amount effective to seal the tear, wherein when exposed to a temperature above its critical solution temperature, the temperature-responsive hydrogel becomes adhesive, and when exposed to a temperature below its critical solution temperature, the temperature-responsive hydrogel becomes less adhesive wherein the temperature-responsive hydrogel includes a polymer disposed on a host material.

In some aspects, the temperature-responsive hydrogel includes a sol recovery rate of about 74 to 99 percent.

In some aspects, the method includes prior to the step of applying and upon cooling the polymer and the host material to a cooling temperature for a cooling duration of time, the temperature-responsive hydrogel becomes homogenous with one or more particles of the polymer stored within pores of the host material, the homogenous temperature-responsive hydrogel including a storage and loss moduli that increases with temperature due to enhanced interpolymer interaction.

In some aspects, the cooling duration of time is between about 5 and 15 minutes.

In some aspects, the cooling duration of time is approximately 10 minutes.

In some aspects, the method includes prior to the step of cooling the polymer and the host material, exposing the polymer and the host material to an exposure temperature for an exposure duration of time between about 1 hour and 24 hours.

In some aspects, the method includes prior to the step of cooling the polymer and the host material, exposing the polymer and the host material to an exposure temperature for an exposure duration of time of at least 1 hour.

In some aspects, the method includes prior to the step of cooling the polymer and the host material, exposing the polymer and the host material to an exposure temperature for an exposure duration of time of at least 24 hours

In some aspects, the tissue is ocular tissue of the cornea, sclera, lens or retina.

In some aspects, the tissue is skin tissue or mucosal tissue.

In some aspects, the polymer is poly(N-isopropylacrylamide-co-butyl acrylate) (pNIPAM).

In some aspects, the polymer is poly(N-isopropylacrylamide-co-N-tert-butylacrylamide) and poly(N-isopropylacrylamide), Poly(N,N-diethyl acrylamide), and poly(methyl vinyl ether), poly(N-vinyl caprolactam), poly(ethylene glycol), poly(propylene glycol), poly(vinylalcohol), poly(N-isopropylacrylamide), poly(methyl vinyl ether), poly(N-vinyl caprolactam) copolymer of poly(ethylene oxide) and poly(propylene oxide) s, and elastin-like oligo- and polypeptides.

In some aspects, a molecular weight of the polymer is at least one of Mn=64 kg mol 1, Mn=41 kg mol 1, Mn=35 kg mol 1, Mn=24 kg mol 1, and Mn=7 kg mol 1.

In some aspects, the host material includes an open-cell foam selected from the group consisting of polyurethane (PUR), polyimide (PIM), hydrophilic polyurethane (PUR), Aquazone® (AQZ), and neoprene (NEO).

In some aspects, the host material includes a foam.

In some aspects, the sol recovery rate is improved by a factor of about 8-10 relative to without the host material.

In some aspects, a gel forming composition is provided that includes an aqueous solution, a host material, a polymer disposed in pores of the host material, wherein the host material and the polymer are suspended in the aqueous solution. Upon exposing the polymer and host material to an exposure temperature that causes the polymer to form a temperature-responsive hydrogel gel and cooling the polymer and the host material to a cooling temperature that causes the temperature-responsive hydrogel gel to transition to a sol.

In some aspects, the temperature-responsive hydrogel including a sol recovery rate of about 74 to 99 percent.

In some aspects, the polymer is poly(N-isopropylacrylamide-co-butyl acrylate) (pNIPAM).

In some aspects, a molecular weight of the polymer is at least one of Mn=64 kg mol 1, Mn=41 kg mol 1, Mn=35 kg mol 1, Mn=24 kg mol 1, and Mn=7 kg mol 1.

In some aspects, the polymer is present in an amount of about 0.5 weight percent to about 50 weight percent of a total weight of the composition.

In some aspects, pores of the host material range between about 0.2-1.1 mm.

In some aspects, the temperature-responsive hydrogel is homogenous with one or more particles of the polymer stored within pores of the host material, the homogenous temperature-responsive hydrogel including a storage and loss moduli that increases with temperature.

In some aspects, the host material includes a foam such as an open-cell foam and a closed-cell foam.

In some aspects, the open-cell foam is selected from the group consisting of polyurethane (PUR), polyimide (PIM), hydrophilic polyurethane (PUR), Aquazone® (AQZ), and neoprene (NEO).

In some aspects, the sol recovery rate is improved by a factor of about 8-10 relative to without the host material.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIGS. 1, 2, and 3 provide a table listing polymers for example implementations of this disclosure.

FIG. 4 illustrates a flowchart for a method, according to an embodiment.

FIG. 5A shows images of changes in physical properties of a temperature-responsive hydrogel and above the critical solution temperature (LCST).

FIG. 5B shows images of reversible phase transition of the temperature-responsive hydrogel of FIG. 5A upon temperature change.

FIG. 6A shows exemplary transitions of temperature-responsive hydrogel of this disclosure from hydrophilic coils to hydrophobic globules.

FIG. 6B shows one example synthesis of temperature-responsive hydrogel of this disclosure.

FIG. 7A shows an example coil-to-globule transition during gelation of a temperature-responsive hydrogel of this disclosure at relatively high temperature.

FIG. 7B shows an example irreversible segregation of the hydrogel of FIG. 7A into two phases upon heating and cooling.

FIG. 8 shows an example representation of enhanced temperature-responsive hydrogel stored within a host material.

FIG. 9A to FIG. 9C specifically show chronological photographs of a syringe loaded with temperature-responsive hydrogel immediately (FIG. 9A), 3 minutes (FIG. 9B), and 6 minutes (FIG. 9C) storage at approximately −40° C. for approximately 1 hour.

FIG. 10A to FIG. 10B show photographs of samples.

FIG. 11 shows scanning electron microscope (SEM) images of example studied host materials.

FIG. 12A shows average recovery of TRS64-10% solution of three replicates with each host material after one hour storage at approximately 50° C. and approximately 10-minute cooling afterwards.

FIG. 12B shows a table providing the average percent recovery and standard deviations relative to a reference host material sample that was not heated.

FIG. 13A shows a graph summarizing exemplary analysis of variance (ANOVA) methods used to determine if the average absorbance values (n=3) at 190 nm for the recovered TRS64-10% solution with each host material after one hour storage at 50° C. followed by 10 minutes of cooling are statistically different.

FIG. 13B shows a graph summarizing prolonged (24 hours) storage at 50° C. compared to 1 hour at 50° C. for samples where the host material is PUR that demonstrate no significant change in improved recovery rate over noF samples.

FIG. 14A shows a graph of viscosity versus shear rate summarizing one-way ANOVA results for prolonged (24 hours) storage at 50° C.

FIG. 14B shows a graph of storage modulus versus temperature for prolonged (48 hours) storage at 50° C. hosted in host material compared to pristine TRS64-10%.

FIG. 15A shows a graph of absorbance versus wavelength of no foam, no heating compared with no foam samples heated at approximately at 50° C.

FIG. 15B shows a graph of absorbance versus wavelength of noFH samples compared with PUR samples heated at approximately at 50° C.

FIG. 16A shows a graph of absorbance versus wavelength of no foam, no heating compared with AQZ foam samples heated at approximately at 50° C.

FIG. 16B shows a graph of absorbance versus wavelength of no foam, no heating compared with PIM foam samples heated at approximately at 50° C.

FIG. 17A shows a graph of absorbance versus wavelength of no foam, no heating compared with PE foam samples heated at approximately at 50° C.

FIG. 17B shows a graph of absorbance versus starting hydrogel concentration in weight percentage.

### DETAILED DESCRIPTION

Disclosed herein are temperature-responsive hydrogel solutions that can be used as sealants in biomedical applications, such as ocular sealants as well as for wound closure. In some aspects, the herein disclosed enhanced hydrogel solutions can be configured for on-demand deployment and be stored at ambient temperature prior to use and then cooled before use. In some aspects, the herein disclosed enhanced hydrogel solutions are formed to include a fast reversible phase transition for higher molecular weight hydrogel that can be achieved with aspects stored within a high surface area host material. In some aspects, the herein disclosed enhanced hydrogel can be a homogenous hydrogel capable of recovering by using host material (e.g., polyurethane foam) after heating for a period of time. Moreover, the gel-sol transition rate of the herein disclosed enhanced hydrogel is configured to increase (e.g., at least eight to ten-fold) relative to hydrogel heated without the host material. The host material can be high surface area foam (e.g., open cell foam) that significantly improves the kinetics of the gel-sol transition of the temperature-responsive hydrogel. In some aspects, by spatially limiting temperature-responsive hydrogel to the foam material, the domain sizes of both phases, including thermoresponsive-rich and pseudoliquid water-rich, are limited and the solvation rate is enhanced.

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise.

In this disclosure, the term “comprising” is synonymous with “including,” “having,” “containing,” or “characterized by.” These terms are inclusive and open-ended and do not exclude additional, unrecited elements or method steps. By using any of these terms, it is meant that at least the named compound, element, particle, or method step is present in the composition or article or method, but does not exclude the presence of other compounds, materials, particles, method steps, even if the other such compounds, material, particles, method steps have the same function as what is named.

In this disclosure, the phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

In this disclosure, the phrase “consisting essentially of” limits the scope of a claim to the specified materials or steps, plus those that do not materially affect the basic and novel characteristic(s) of the claimed subject matter.

In this disclosure, where publications are referenced, the disclosures of these publications in their entireties are hereby incorporated by reference into this application.

In this disclosure, relative terms, such as “about,” “substantially,” or “approximately” are used to indicate a possible variation of +10% in the stated value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

In this disclosure, the following abbreviations are used:

“LCST” means lower critical solution temperature.

“UCST” means upper critical solution temperature.

“TRS” means thermoresponsive sealants, such as but not limited to temperature-responsive hydrogel.

Except in the examples, or where otherwise expressly indicated, all R groups (e.g., R and Rᵢ where i is an integer) include alkyl, lower alkyl, C₁₋₆ alkyl, C₆₋₁₀ aryl, or C₆₋₁₀ heteroaryl; single letters (e.g., “m” “n” or “o”) are 1, 2, 3, 4, or 5; ranges of integers specifically include individual the endpoints and all intervening integers (e.g., 1-5 specifically includes each of 1, 2, 3, 4, and 5); percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

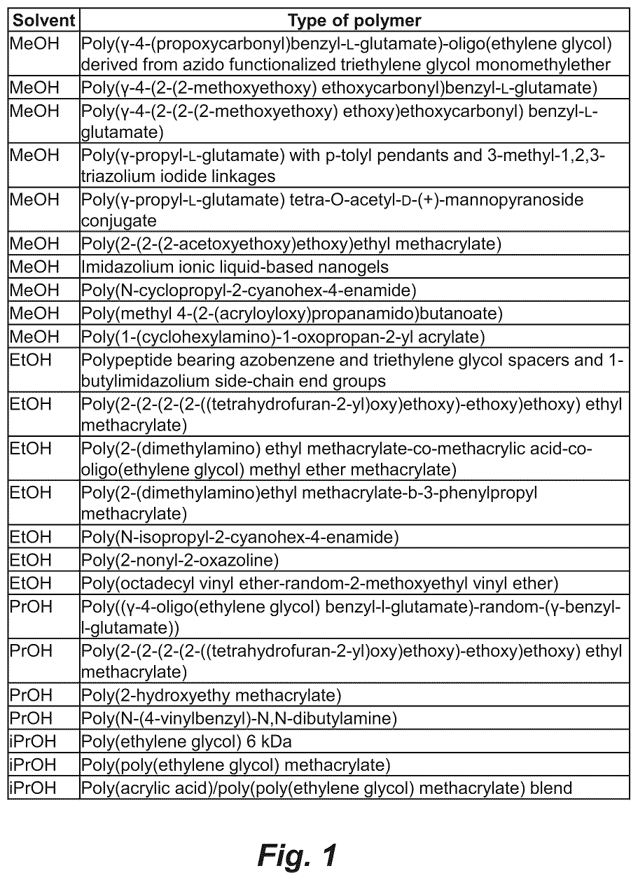

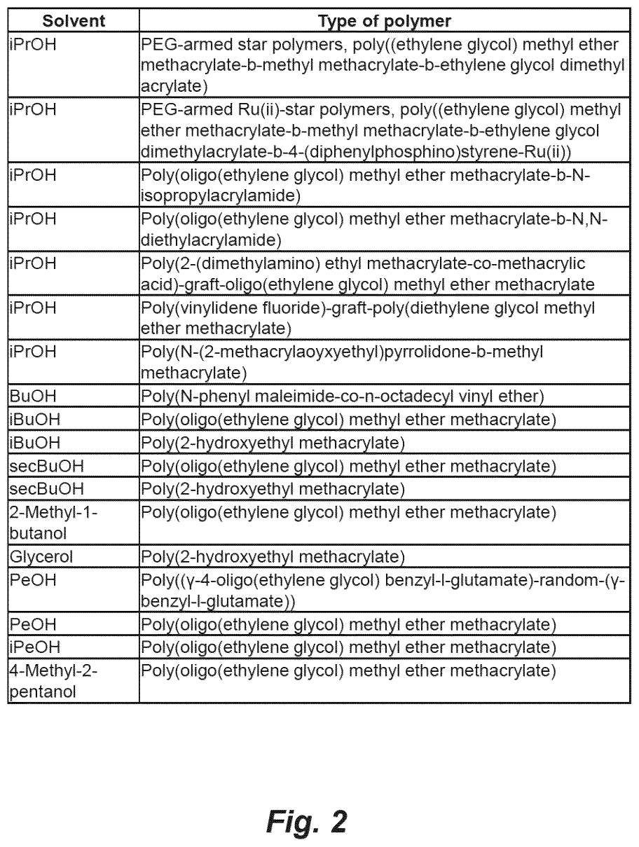

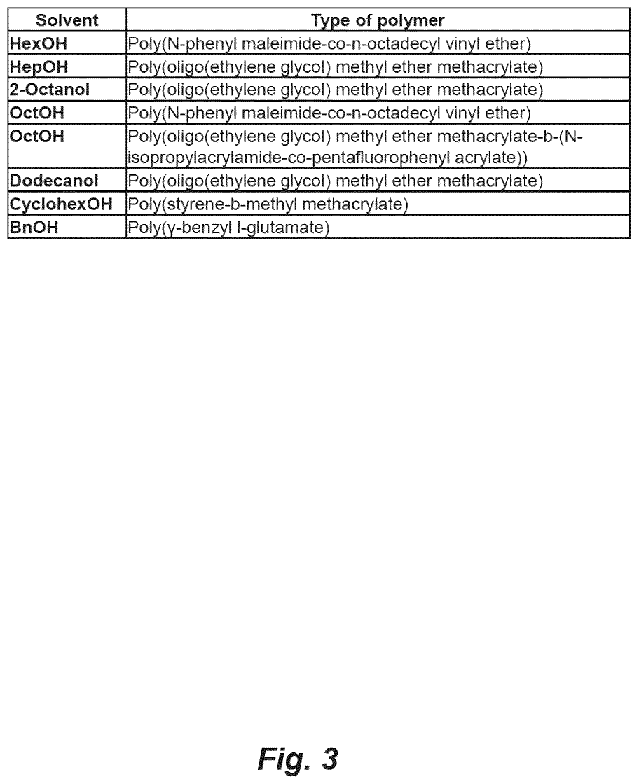

In an embodiment, a temperature-responsive hydrogel of this disclosure can include at least include an aqueous solution and a polymer (e.g., poly(N-isopropylacrylamide-co-butyl acrylate)) (hereafter “pNIPAM”). While pNIPAM is discussed throughout this disclosure, the herein disclosed solution could be used with any number of other thermoresponsive polymers, such as NIPAM-based homo- and copolymers including poly(N-isopropylacrylamide-co-N-tert-butylacrylamide) and poly(N-isopropylacrylamide), poly(N,N-diethyl acrylamide), and poly(methyl vinyl ether), poly(N-vinyl caprolactam), poly(ethylene glycol), poly(propylene glycol), poly(vinylalcohol), poly(N-isopropylacrylamide), poly(methyl vinyl ether), poly(N-vinyl caprolactam), copolymer of poly(ethylene oxide) and poly(propylene oxide) s, and elastin-like oligo- and polypeptides, and warm release adhesives such as gelatin, Poloxamer 407, and κ-carrageenan using any open cell with void sizes between approximately 0.05 and 3 mm. Other polymers contemplated for use in aspects of this disclosure can include those listed in Leda Klouda, et al., Thermoresponsive hydrogels in biomedical applications, European Journal of Pharmaceutics and Biopharmaceutics, Volume 68, Issue 1 (2008) at pages 34-45, which is incorporated by reference herein in its entirety as if set forth verbatim. FIGS. 1 to 3 provide a table showing examples of polymers contemplated for use in examples of this disclosure that demonstrate phase transition in alcoholic solvents. For example, the temperature-induced phase transitions of synthetic polypeptides in alcohols and, in particular, in methanol showed promising result for their potential application in the biomedical field as smart material.

In some aspects, the temperature-responsive hydrogel can be disposed on a host material such as a foam, as further discussed in this disclosure. In some aspects, the polymer and aqueous solution can include any particular amount for its components. In one embodiment the polymer is nPIPAM and is present in an amount of about 0.5 weight percent to about 50 weight percent of the total weight of the temperature-responsive hydrogel. In another variation, the nPIPAM is present in an amount of about 10 weight percent to about 60 weight percent of the total weight of the temperature-responsive hydrogel.

Although the present disclosure is not significantly limited by the molecular weight of the polymer, the nPIPAM can include a number average molecular weight of about 5,000 to about 5,000,000 Daltons. In some aspects, the nPIPAM can include a number average molecular weight of about 10,000 to about 3,000,000 Daltons. In some aspects, the nPIPAM can include a number average molecular weight of about 20,000 to about 2,000,000 Daltons. In some aspects, the nPIPAM can include a lowest molecular weight of about 15,000 to about 1,000,000 Daltons.

In some aspects, the temperature-responsive hydrogel of the present disclosure can be used as an ocular sealant (e.g., reversibly sealing an ocular perforation) by being applied to seal a tear in tissue of a subject, where the tissue can include ocular tissue (e.g., tissue of the cornea and sclera), skin, mucosal tissue, wound tissue, etc. The temperature of the recipient tissue is usually above the critical solution temperature of the temperature-responsive hydrogel to facilitate adherence to the tissue. In some aspects, the enhanced temperature-responsive hydrogel of the present disclosure when disposed on or otherwise with the host material can be in a liquid state and applicable at one temperature, and transition to a solid state at the target temperature. In some aspects, the polymer(s) of the temperature-responsive hydrogel of the present disclosure includes a coil-to-globule transition above its lower critical solution temperature. In some aspects, the polymer can be PNB5% (e.g., a copolymer of 95% N-isopropylacrylamide and 5% butyl acrylate) that has an LCST of approximately 24° C., which is below the human body temperature. Keeping the LCST below body temperature can be important to maintain adhesion of the sealant to the wound.

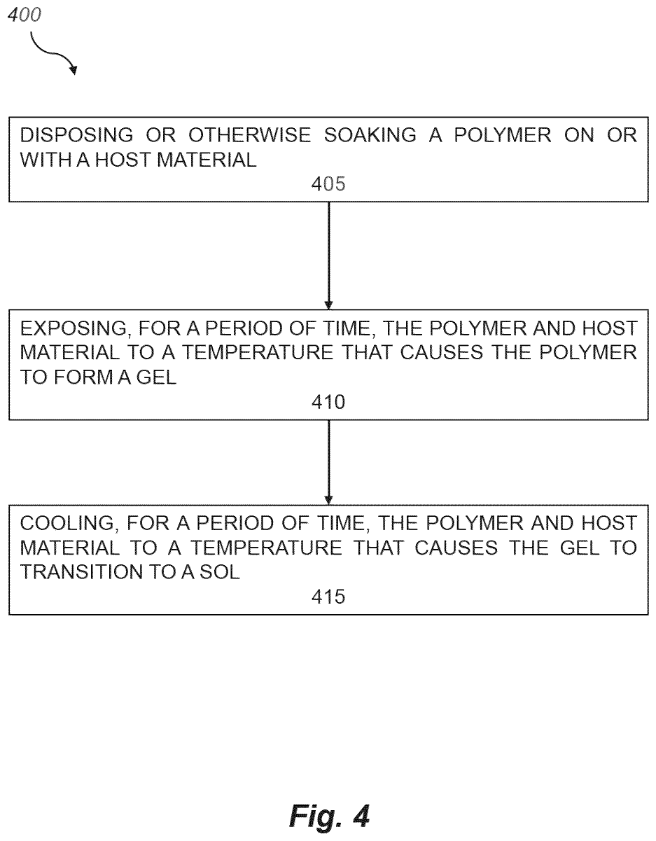

In some aspects, a method 400 is disclosed in FIG. 4 for enhancing a gel to sol transition of a polymer (e.g., a biocompatible thermoresponsive sealant). Method 400 can include step 405 of disposing or otherwise soaking the polymer on or with a host material, such as foam. In some aspects, the foam can be open-cell foam such as polyurethane (PUR), polyimide (PIM), hydrophilic polyurethane (PUR), Aquazone® (AQZ), though other open-cell foams are contemplated as needed or required. In some aspects, the foam can be a closed-cell foam such as neoprene (NEO) and polyethylene (PE). In some aspects, pores of the foam can range between approximately .2 to 1.1 mm.

Method 400 can include step 410 of exposing, for a period of time such as between approximately 1 hour and 24 hours, at least 1 hr, at least 24 hours, etc, the polymer and host material to a temperature that causes the polymer to form a gel (e.g., a temperature that is similar to relatively hotter storage like temperatures). In some aspects, the period of time can be longer than 24 hours (e.g., 48 hours, 72 hours, 96 hours, 7 days, etc.). In some aspects of step 410, the temperature can range from about 25 to 75° C. In some aspects of step 410, the temperature can be approximately 50° C.

Method 400 can include step 415 of cooling, for a period of time, the polymer and host material to a temperature that causes the gel to transition to a sol. In some aspects of step 410, the period of time can be approximately 10 minutes though other times shorter (e.g., less than 10 minutes such as 5 minutes, 4 minutes, 3 minutes, 2 minutes) and longer (e.g., greater than 10 minutes such as 15 minutes, 20 minutes, etc.) are contemplated. In some aspects, during the step 415 of cooling the temperature is less than about 25° C. In some aspects, during the step 415 of cooling the temperature is from about −40 to 20° C. In some aspects, during the step 415 of cooling the temperature is from approximately 0° C.

In some aspects, upon step 415 an enhanced hydrogel is formed that is substantially homogenous. In some aspects, the polymer of the enhanced hydrogel can be stored within pores of the host material. In some aspects, where the polymer of the enhanced hydrogel is pNIPAM, its molecular weight can include a number that includes Mn=64 kg mol⁻¹, PDI=3.24 (TRS64), Mₙ=41 kg mol⁻¹, PDI=3.00 (TRS64), Mₙ=35 kg mol⁻¹, PDI=2.84 (TRS35), Mₙ=24 kg mol⁻¹, PDI=1.99 (TRS24), and Mₙ=7 kg mol⁻¹, PDI=3.29 (TRS7). In some aspects, the sol recovery rate resulting from step 415 with the polymer disposed on or otherwise with the host material of this disclosure is from about 74 to 99 percent. In some aspects, the sol recovery rate resulting from step 415 with the polymer disposed on or otherwise with the host material of this disclosure is at least 93 percent. In some aspects, the sol recovery rate resulting from step 415, with the polymer disposed on or otherwise with the host material being PU, is approximately 100 percent. In some aspects, the sol recovery rate is enhanced by a factor of approximately 8-10 when the host material is present versus if no host material is present.

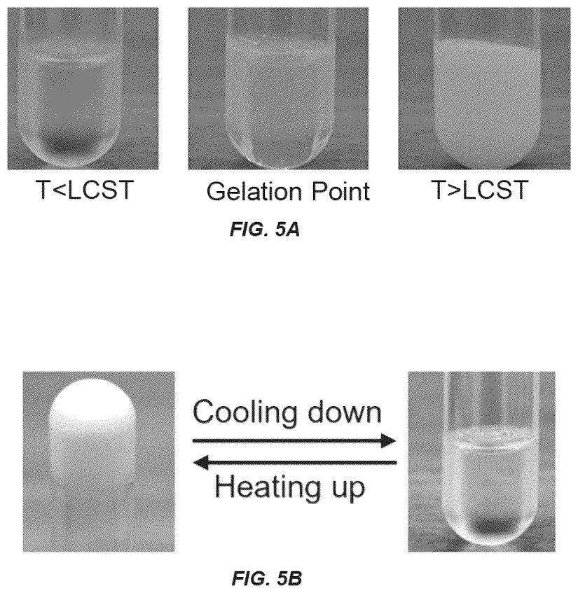

FIG. 5A shows images of changes in physical properties of a temperature-responsive hydrogel of this disclosure and above its critical solution temperature (LCST). In particular, the left portion of FIG. 5A shows a picture of temperature-responsive hydrogel where its temperature is less than the LCST. The central portion of FIG. 5A shows a picture of temperature-responsive hydrogel at its gelation point. The right portion of FIG. 5A shows a picture of temperature-responsive hydrogel where its temperature is greater than the LCST. FIG. 5B shows images of reversible phase transition of the temperature-responsive hydrogel of FIG. 5A upon temperature change from heating up (left view) to cooling down (right view). At high concentrations and/or degrees of polymerization this the temperature-responsive hydrogel of this disclosure are configured to undergo an inverse-phase transition, meaning, its aqueous solution is a free-flowing liquid at low temperature (e.g., approximately ≤10° C.), and the polymer (e.g., pNIPAM) of the enhanced hydrogel goes under sol-to-gel transition upon warming to higher temperatures (e.g., approximately 32° C.), such as in FIG. 5A. In some aspects, when storage temperatures exceed 25° C., the polymer (e.g., pNIPAM) forms a suspension in the aqueous solution (e.g., water) with semi-solid-hydrated polymer suspended in dilute solution, as in FIG. 5B. Due to similarities between the LCST of example temperature-responsive hydrogels of this disclosure, such as pNIPAM, and human body temperature, temperature-responsive hydrogels can be applied to aspects such as wound dressings as a free-flowing solution and subsequently forming a gel and sealing a wound upon tissue contact at body temperature.

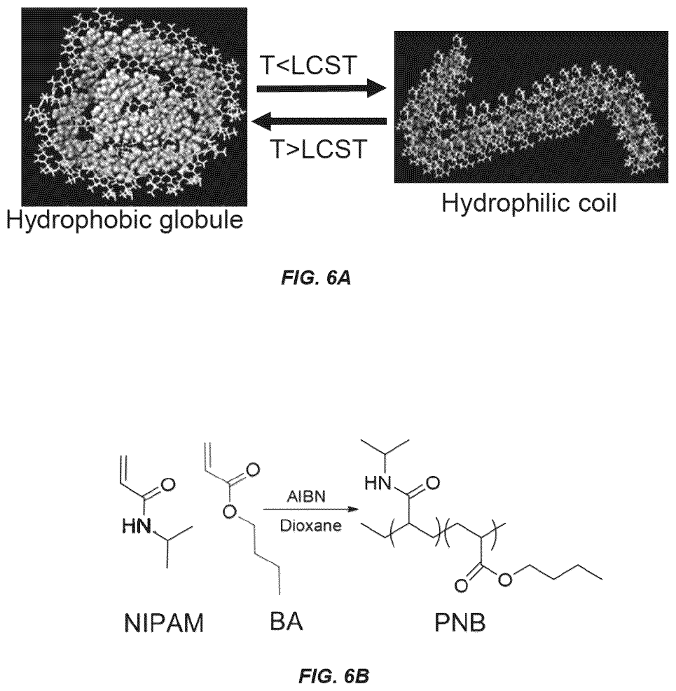

FIG. 6A shows exemplary transitions of temperature-responsive hydrogel of this disclosure from hydrophilic coils to hydrophobic globules. FIG. 6B shows one example synthesis of temperature-responsive hydrogel of this disclosure. In the example synthesis of FIG. 6B, copolymerization of the illustrated pNIPAM with butyl acrylate (which forms PNB) acts to lower the LCST, giving a sharper and more rapid transition between the solution and gel phases. In some aspects, PNB5% (e.g., a copolymer of 95% N-isopropylacrylamide and 5% butyl acrylate) has an LCST of 24° C., well below the human body temperature. In some aspects, keeping the LCST below body temperature can be important to maintain adhesion of the example sealant of this disclosure to the wound. In some aspects, PNB5% can be used as a wound dressing for open globe injuries. In some aspects, PNB5% can exhibit non-adhesive behavior at cold temperatures. At body temperature, PNB5% can become viscous and adhesive and is capable of sealing penetrating injuries.

The following examples illustrate the various embodiments of the present disclosure. Those skilled in the art will recognize many variations that are within the spirit of the present disclosure and scope of the claims.

This section reports the design and fabrication of a novel system for using host material(s) (e.g., high surface area foams) with polymer(s) that improve kinetics of gel-sol transition in temperature-responsive hydrogel.

In a first example, the host materials investigated included open-cell foams including polyimide (PIM), polyethylene (PE), hydrophilic polyurethane (PUR), Aquazone® (AQZ), and neoprene (NEO). Poly(NIPAM-co-butylacrylate 95:5) was synthesized in different number average molecular weights: Mₙ=64 kg mol⁻¹, PDI=3.24 (TRS64), Mₙ=41 kg mol⁻¹, PDI=3.00 (TRS64), Mₙ=35 kg mol⁻¹, PDI=2.84 (TRS35), Mₙ=24 kg mol⁻¹, PDI=1.99 (TRS24) and Mₙ=7 kg mol⁻¹, PDI=3.29 (TRS7).

To prepare samples, a cylindrically cut foam sample was loaded into a 1 mL syringe. Considering the differing volume fractions of the foams, each foam sample was optimized to confine or soak up approximately 0.6 mL of TRS64-10% solution in a 1 ml syringe. The syringe was sealed with a septum, and a vacuum of ca. 0.3 Torr was applied to the sealed syringe. The cold TRS64-10% (0.6 mL) at atmospheric pressure was then drawn into the syringe through the septum and the filled syringe placed in a water-ice bath (ca. 0° C.). The syringe was placed in an oven at approximately 50° C. for a fixed period (e.g., approximately 1 or approximately 24 hours) to simulate storage above ambient temperature, then cooled in a water-ice bath for approximately 10 minutes.

The contents of the syringe were then expelled to obtain the recovered hydrogel solution for analysis. For samples without foam (noF), syringes were weighed, then filled with approximately 0.6 mL of the cold hydrogel solution under ambient pressure and reweighed. The syringes were heated in an oven at approximately 50° C. for approximately 1 hour, then cooled in an ice bath at approximately 0° C. for approximately 10 minutes. The contents of the syringes were subsequently ejected, and the emptied syringes were reweighed. Reference experiments (e.g., no foam-no heat, noFH) were performed with syringes loaded with cold hydrogel under ambient pressure and stored in a water-ice bath until use. During the experiment, they were stored at ambient temperature (no heat) for 1 hour before cooling in a water ice-bath.

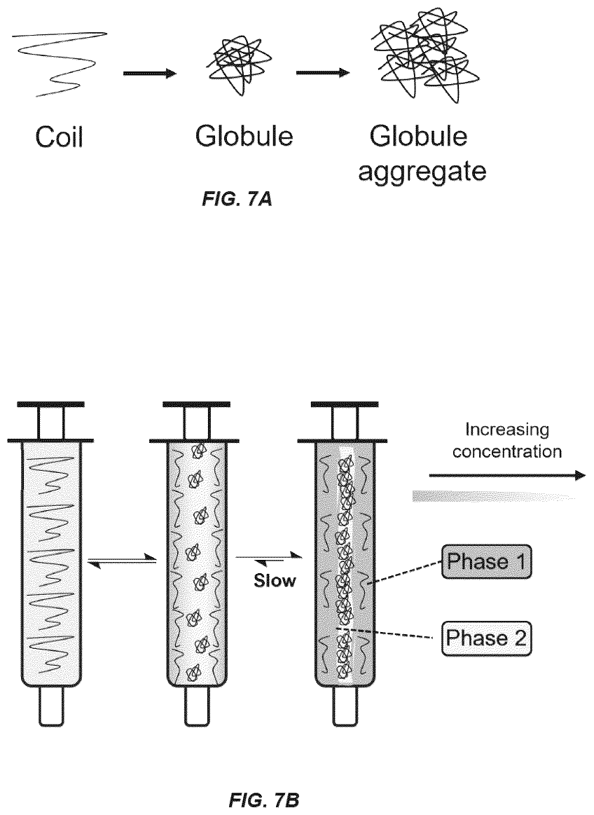

Aspects of sample preparation are shown in FIGS. 7A, 7B, and 8. Specifically, FIG. 7A shows an example coil-to-globule transition during gelation of a temperature-responsive hydrogel that includes pNIPAM at high relatively high temperature, including the illustrative transition from coil (left view) to globule (central view) to globule aggregate (right view). FIG. 7B shows an example irreversible segregation of temperature-responsive hydrogel solution into two phases upon heating and cooling. Phase 1 represents a dilute solution that includes pNIPAM, whereas phase 2 represents a partially gelated and dehydrated pNIPAM-rich phase. The sol-gel transition of FIG. 7B leads to two segregated pseudo-liquid phases, as shown, a partially dehydrated, TRS-rich phase (phase 2) and an external shell of dilute TRS solution (phase 1). Upon cooling the temperature-responsive hydrogel, the TRS-rich phase redissolves, forming a homogeneous solution. The phase transition upon cooling is reversible and rapid (e.g., approximately <5 min.) when the depicted sample is stored at ambient temperature. However, exposure to higher temperatures (e.g., approximately 50° C.), as may be experienced in storage and shipping, results in an either irreversible or slow (approximately >1 hour) gel-sol transition on colling to approximately <10° C.

FIG. 8 shows an example representation of enhanced temperature-responsive hydrogel of this disclosure stored within host material (e.g., foam). In the graphic of FIG. 8, the polymer of the enhanced temperature-responsive hydrogel is pNIPAM whereby the host material limits the domain size of high- and low-density polymer phases. By limiting the domain size, the rate gel-sol transition dynamics and reversibility are enhanced. In some aspects, the enhanced temperature-responsive hydrogel achieves reversible gel-sol transition via utilization of the host material (e.g., a high surface area open-cell foam host material), shown pictorially in FIG. 8. In some aspects, the use of the high surface area, open-cell foams can markedly improve the kinetics of the gel-sol transition of the temperature-responsive hydrogel, particularly when used as a sealant (e.g., as an ocular sealant). By spatially limiting temperature-responsive hydrogel to the host material, the domain sizes of both phases (TRS-rich and pseudo-liquid water-rich) are limited and, consequently, the solvation rate is enhanced.

Ultraviolet-visible analysis (UV-vis) was also performed whereby the UV-vis absorption spectra were measured from approximately 190 to approximately 700 nm using a UV-vis diode array spectrophotometer. Recovered samples from each syringe were diluted to approximately 1 L with deionized (DI) water in a 1 L volumetric flask. Approximately 100 mL aliquot of the dilute solution was further diluted to approximately 500 mL with DI water in volumetric flask. To calculate mass recovery, the mass of temperature-responsive hydrogels ejected from the syringe was divided by the mass of temperature-responsive hydrogel initially loaded into the syringe.

The rheological properties were also analyzed with a hybrid rheometer equipped with a 20-mm parallel plate with a 0.5 mm gap with a solvent trap to minimize water evaporation. For flow sweep experiments, the temperature was held at approximately 10° C. with increasing shear rate from 1 s⁻¹ to 500 s⁻¹ with a logarithmic sweep mode. Temperature ramp scans were performed with the frequency held at 6.3 rad s⁻¹, strain at approximately 1% and temperature scanned in the range of approximately 10-50° C. with a heating rate of approximately 1° C. min-1.

Samples for scanning electron microscopy (SEM) imaging were cut out in approximately 3 mm×3 mm squares from randomly chosen locations on foam sheets with a sharp blade, then sputter coated with palladium. To estimate the pore size of each foam, micrographs of the foam surface were acquired with a scanning electron microscope using a spot size of 3 nm and an accelerating voltage of 3 kV. To study how the temperature-responsive hydrogels behave in the presence of the PUR foam, one syringe was loaded with foam and TRS64-10%, then placed in the oven at approximately 50° C. for approximately 1 hour. The contents of the syringe were lyophilized. A disc was cut out of the hydrogel filled foam, sputter coated with palladium, and scanned with SEM.

Displayed data are reported as mean±standard deviation. Grouped analyses were conducted using analyses of variance (ANOVA) testing. Significant ANOVA results underwent multiple comparison post-hoc testing. In the ANOVA analysis, p-values are used to describe the probability that the observed difference between two data sets is due to random chance. The closer the p value is to 0, the higher the confidence level is that two data sets are not statistically different. In graphical representations of data, values of p<0.001 denoted by ***, and p<0.0001 denoted by ****(no values of p>0.001 were observed).

With respect to results of this example, PNB5% samples with a range of number average molecular weights (Mₙ=7-64 kg mol⁻¹) and in different concentrations (10-40 wt. %) were used for a qualitative study of how the concentration and molecular weight (MW) of the temperature-responsive hydrogel affect the kinetics of the gel-sol phase transitions. The samples are denoted by an acronym of TRS (e.g., to signify the temperature-responsive hydrogel) followed by Mₙ and finally the concentration; or example TRS35-10% is a 10% solution of a TRS with a Mₙ of 35 kg mol⁻¹.

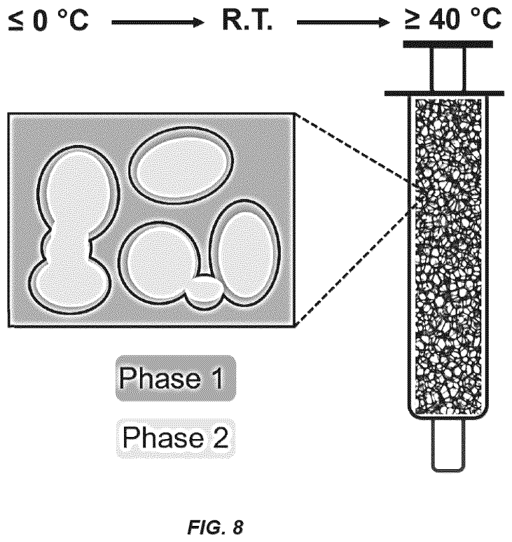

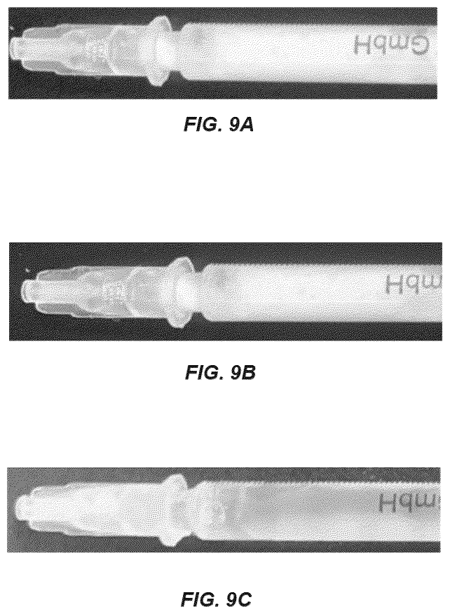

TRS35-10% and TRS64-10% were first compared to assess recovery as a function of the molecular weight of the hydrogel polymer. A concentration of 10% was chosen because the viscosities of the solutions are suitable as ocular sealants. In some aspects, the temperature-responsive hydrogel needs to be ready to apply within a few minutes of cooling to approximately 10° C. and be stable in extreme environments, such as extreme heat and cold. The stability of both TRS35-10% and TRS64-10% were examined at extremely cold temperatures (−40° C.), as shown in FIGS. 9A to 9C. Specifically, after storage at approximately −40° C. for approximately 1 hour, warming to room temperature for approximately 10 minutes made the TRS64-10% suitable for injection into the eye as a sealant. The temperature-responsive hydrogel was frozen and melted slowly until returning to its original fluid state in approximately 6 minutes. FIG. 9A to FIG. 9C specifically show chronological photographs of a syringe loaded with temperature-responsive hydrogel immediately (FIG. 9A), 3 minutes (FIG. 9B), and 6 minutes (FIG. 9C) storage at approximately −40° C. for approximately 1 hour. The same experiment was done on TRS35-10%. The temperature-responsive hydrogels freeze within the syringes but warming to ambient temperature for 10 minutes made the samples suitable for injection into the eye as a sealant.

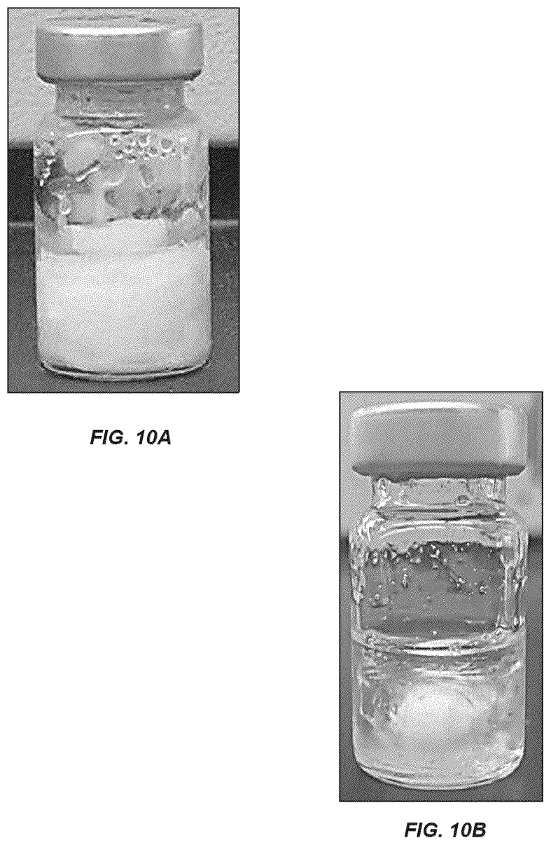

The propensity for heterogeneous phase separation of the temperature-responsive hydrogels upon heating to temperatures >30° C. presents potential problems for storage in field-based applications, limiting the rate of achieving the phase transition to the solution phase. This is accentuated when pNIPAM solutions are heated to temperatures well above ambient temperature, as can arise during transport and storage. The results here as to PNB copolymers for wound sealant is consistent with the core-corona structure seen for pNIPAM, as shown previously in FIG. 7B. It was observed that solutions of PNB stored at approximately 50° C. resulted in two separate phases: a heavily diluted, free-flowing PNB solution (e.g., possessing lower viscosity than the original PNB solution) which forms a shell around a core of dehydrated PNB gel, as shown in FIGS. 10A to 10B. Specifically, FIGS. 10A to 10B show photographs of these samples after exposure to higher temperatures (e.g., 50° C.), such as may be experienced in storage and shipping, results in slow (e.g., >24 hours) gel-sol transition on cooling to temperatures approximately lower than 10° C. FIG. 10A shows a photograph of the TRS64-10% after storage at 5 approximately 0° C. for 1 hour, whereby solutions of PNB stored at approximately 50° C. resulted in two separate phases: a heavily diluted, free-flowing PNB solution (possessing lower viscosity than the original PNB solution) which forms a shell around a core of dehydrated PNB gel. FIG. 10B shows a photograph of the same sample of FIG. 10A, which was not completely redissolved, after cooling for approximately 10 minutes.

Temperature-responsive hydrogels were then studied at elevated temperatures. Samples at a concentration of 10% wt. with Mn<41 (kg mol⁻¹) were heated to 50° C. for 1 hour followed by cooling in a water-ice bath for 10 minutes and found to return to a free-flowing state with the same properties as a temperature-responsive hydrogel sample that was not heated. However, 10% wt. temperature-responsive hydrogel solutions with Mn≥41 kg mol⁻¹ showed a degree of aggregation and precipitation severe enough to significantly retard the gel-sol transition, leading to a biphasic sample after the same heating and cooling regiment. Concentration above 10% for all of the temperature-responsive hydrogel studied here (Mn=7-64 kg mol⁻¹) gave nonhomogeneous samples after heating to 50° C. for 1 hour and cooling in a water-ice bath for 10 minutes, as shown here:

| Sample | | | | | | | | | | | |
|---|---|---|---|---|---|---|---|---|---|---|---|
| (Mₙ, kg mol⁻¹ ) | 7 | | | | 24 | | | 35 | | 41 | |
| Concentration | 10 | 20 | 30 | 40 | 10 | 20 | 30 | 10 | 20 | 10 | 20 |
| (w/w %) | | | | | | | | | | | |
| Mass recovery(%) | 91 | 76 | 49 | 6 | 90 | 83 | 34 | 92 | 74 | 79 | 45 |

Each syringe here was filled with approximately 0.6 mL of hydrogel solution and heated in an oven at approximately 50° C. for approximately 1 hour, then cooled in an ice bath at approximately 0° C. for approximately 10 minutes. Samples at a concentration of approximately 10% wt. with Mn<41 (kg mol⁻¹) were heated to approximately 50° C. for approximately 1 hour followed by cooling in a water-ice bath for 10 minutes and found to return to a free-flowing state with the same properties as a hydrogel sample that was not heated. However, 10% wt. hydrogel solutions with Mn≥41 kg mol⁻¹ showed a degree of aggregation and precipitation severe enough to significantly retard the gel-sol transition. Concentration above 10% for all of the TRS gave nonhomogeneous samples after heating to approximately 50° C. for approximately 1 hour and cooling in a water-ice bath for approximately 10 minutes. A highly dilute hydrogel solution was obtained with a viscous, hydrogel-rich phase remaining inside the syringe. The mass recovery of each sample was calculated by dividing the mass of hydrogel solution ejected from the syringe by the mass of initial hydrogel solution loaded into the syringe.

To study the cooling time to promote the gel-sol transition for higher molecular weight hydrogel samples, TRS64-10% solutions was heated to approximately 50° C. for approximately 1 hour and cooled for approximately 10 minutes and expelled from the syringe. A highly dilute hydrogel solution was obtained with a viscous, hydrogel-rich phase remaining inside the syringe. Further cooling the heated sample of TRS64-10% in a water-ice bath up to approximately 24 hours improved recovery, but still did not lead to a homogeneous solution, showing that rate of the gel-sol transition is a problem for higher molecular weight hydrogel materials and higher concentrations of lower molecular weight hydrogel on exposure to high temperature.

It was observed that a high surface area host increased recovery of TRS64-10% samples after heating and cooling by limiting bulk segregation via decreased physical domain size. Heating the PUR-hosted system to approximately 50° C. leads to a same transformation, but the dehydrated hydrogel is distributed over the foam surface. The smaller particles of the hydrogel can go through the gel-sol transition much more rapidly than a large mass of the same material without the host material. To assess this approach, TRS64-10% solutions were hosted in PUR foam and heated at approximately 50° C. for approximately 1 hour, then cooled in a water-ice bath at approximately 0° C. for approximately 0, approximately 2, approximately 5, and approximately 10 minutes. The gel-sol transition progressed with extended cooling time, leading to a homogeneous solution and nearly quantitative approximately 93% recovery of the hydrogel solution after approximately 10 minutes at approximately 0° C.

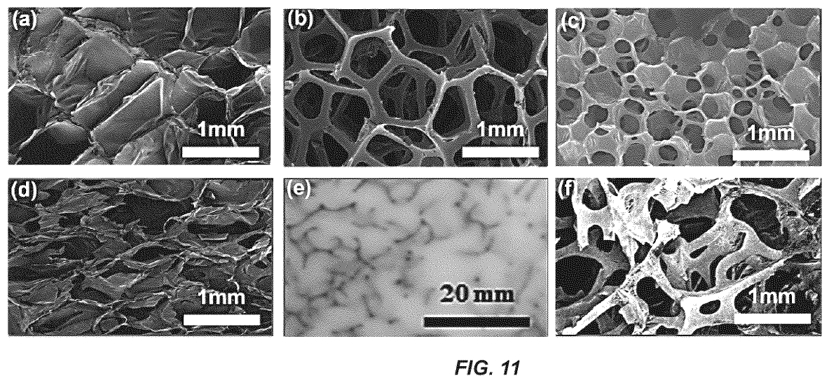

The percent recovery of TRS64-10% solutions was compared in the presence of a high surface area host foam for five foam types: PIM, PE, PUR, AQZ, and NEO, as shown in FIG. 18 which shows SEM images in views (a) to (f) of these example studied host materials. Specifically, SEM images of foams for shown in view (a) polyethylene, PE (closed-cell), view (b) polyurethane, PUR (520-990 μm), view (c) Aquazone®, AQZ (180-380 μm), view (d) polyimide, PIM (220 μm-1.13 mm), view (e) which shows TRS64-10% solidified at approximately 50° C. and hosted in PUR foam, view (f) shows TRS64-10% hosted within the PUR pores after heating to approximately 50° C., then freeze-drying. In views (a) to (d) of FIG. 11, photographs show the pores of the four foams used in this study, which are in the approximately 0.2-1.1 mm range. After inserting a fitted, cylindrical piece of a selected foam into a 1 mL syringe, vacuum was applied to the syringe to remove air from the pores of the foam to ensure homogeneous loading of hydrogel solutions. If the loading is done at ambient pressure, a substantial amount of air remains trapped in the foam. Each syringe was loaded with a fixed amount of TRS64-10% solution precooled in a water-ice bath. After heating the syringe to approximately 50° C. for approximately 1 hour, it was cooled in an ice bath for approximately 10 minutes prior to hydrogel recovery analysis.

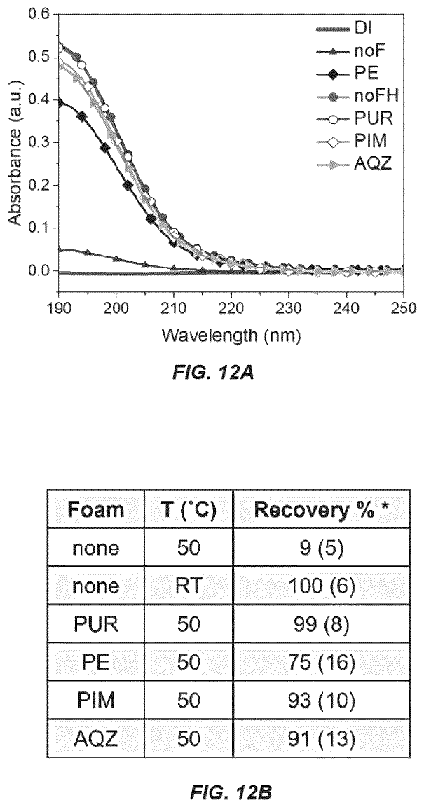

NEO visibly showed separate foam and hydrogel phases, while the materials showed good soaking of the hydrogel solution into the foam. All of the host materials improved the rate of the gel-sol transition and resulted in a homogenous solution of the hydrogel expelled from the syringe after approximately 10 minutes of cooling in the ice bath, as shown in FIG. 12A. The percent recovery of these samples was compared against two controls: one sample subjected to the same heating and cooling cycles as the prior samples but without a foam host (hereafter “noF”), and a second control that was not subject to heating (i.e . . . , no foam and no heat and hereafter “noFH”). The noFH control represents the best possible recovery expected with storing the sample at ambient temperature followed by approximately 10 minutes cooling, given unavoidable losses of material in the syringe.

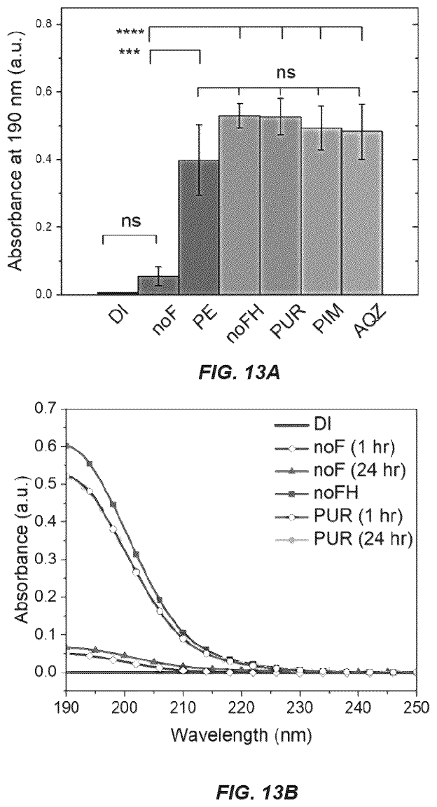

The percent hydrogel recovered upon cooling was investigated via UV-Vis absorption spectroscopy, using the absorption of the amide group at approximately 190 nm to follow the recovery of the hydrogel expelled from the syringe after cooling. The absorption spectra of three samples were averaged for each foam and are plotted in FIG. 12A. FIG. 12B shows the percent recovery for each of these samples, calculated by dividing the averaged absorption of each sample at 190 nm by that of noFH samples. The UV-Vis analysis demonstrates enhanced recovery of the hydrogel by a factor of approximately 8-10 when a foam host is present in the syringe, even for the closed-cell PE foam. Exemplary ANOVA examination is shown in FIG. 13A. Specifically, FIG. 13A shows the ANOVA methods used to determine if the average absorbance values (n=3) at approximately 190 nm for the recovered TRS64-10% solution with each host material after one hour storage at approximately 50° C. followed by approximately 10 minutes of cooling are statistically different from each other or the reference samples. In FIG. 13A, the denoted “ns” means that a value is not statistically different while p<0.001 is denoted by ***, and p<0.0001 by ****. In FIG. 13A, DI (deionized) water shows the absorbance in the pure solvent at 190 nm.

In the study, the kinetics of the gel-sol transition of different concentrations of aqueous solutions with PNB5% were investigated with a number average molecular weights in the range of approximately 7 to 64 kg mol⁻¹. In some aspects, when samples with Mn≥41 kg mol⁻¹ hydrogel at 10% wt. solution were heated at approximately 50° C. for approximately an hour, the gel-sol transition was not reversible after cooling for over approximately 24 hours. In contrast, heating samples with Mn<35 kg mol⁻¹ polymer to approximately 50° C. for approximately an hour gave as a homogeneous solution when cooled for 10 minutes. For higher concentrations than 10% wt, all the Mn TRS solutions studied showed low rate of gel-sol transition.

Based on the foregoing ANOVA examination, it was observed that there is no statistical difference between the sample recovery of hydrogel in the presence of the host material of high surface area foams studied and the noFH reference, demonstrating that all foams effectively improved the kinetics of the gel-sol transition and hydrogel recovery. The closed-cell PE foam gave a high standard deviation, and the mean is well below the open-cell foams, leading to its lower p value. In the studied range of pore sizes (approximately 0.2-1.1 mm), host materials with different void sizes show insignificant differences in the recovery of the hydrogel on cooling (see FIG. 13B). PUR was further investigated because it demonstrated highest hydrogel recovery at nearly 100% and was available in medical grade. Mass recovery was used to study the recovery of free-flowing TRS after heating. “Mass recovery” as used in this disclosure means total material expelled from a 1-mL syringe after following the same heating/cooling procedure discussed previously. Mass recovery data shows that the noFH mass recovery (92±4%) and PUR mass recovery (93±7%) are nearly identical. Furthermore, the noF samples show a lower mass recovery (77±2%) and TRS recovery (9.1%), which reflects the highly dilute nature of the solution recovered after heating/cooling cycles.

To investigate the morphology of the hydrogel within the PUR foam, SEM micrographs were obtained of TRS64-10% freeze-dried within the PUR host as well as PUR-hosted TRS64-10% dried at ambient temperature and low pressure. Both samples were subject to heating for approximately 1 hour at 50° C. Samples that were lyophilized (See view (f) of FIG. 11) demonstrate a uniform distribution of the hydrogel solution in the pores.

To further investigate the stability of enhanced storage within PUR foam, the above procedure was repeated and compared to noF and PUR samples heated at approximately 50° C. for approximately 24 hours. FIG. 13B shows that the hydrogel recovery for these samples and those heated to approximately 50° C. for only one hour are essentially identical, further supporting the viability for longer storage times at high temperature. The results of FIGS. 13A to 13B illustrate that there is no significant difference between the absorbance of noF control and DI, showing that solution recovered from noF is effectively DI water and that the hydrogel remained unrecovered within the syringe. The improved rate for the gel-sol transition of the hydrogel with a high surface area foam in the syringe was confirmed with the significant difference between the absorbances at 190 nm of noF and the other samples.

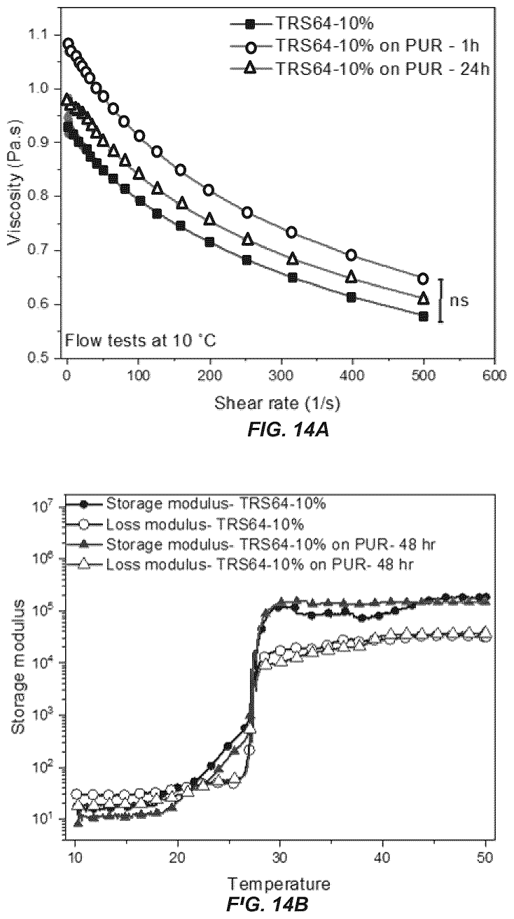

To confirm that the physical properties of the hydrogel were not significantly affected upon high temperature storage within the foam matrix, flow-sweep rheological analysis was performed on pristine TRS64-10% solutions, those stored in PUR for approximately 1 hour, and PUR for approximately 24 hours. FIG. 14A shows a graph of viscosity versus shear rate for prolonged (24 hours) storage at 50° C., which demonstrates no significant (one-way ANOVA, n=3) change in recovery when compared to storage at approximately 1 hour at approximately 50° C. in host material of PUR foam and noFH samples. The flow tests were carried out at 10° C. FIG. 14A shows that the viscosity of samples stored in PUR were not significantly altered by heating.

Temperature ramp studies were also performed to ascertain if the LCST of the hydrogel is affected by storage in a host material of PUR foam. Storage and loss moduli are shown in FIG. 14B for a noFH sample and a sample which was stored in PUR heated to 50° C. for 48 hours. Dynamic modulus measurements were used to study phase transitions, which determine the response of recovered solution to an oscillatory force (stress) or deformation (strain). The viscoelastic properties of TRS64-10% and TRS64-10% heated and cooled with a PUR host foam were studied by measurements of storage modulus G′ (elastic behavior) and loss modulus G″ (viscous behavior). The oscillation temperature ramp of FIG. 14B shows that in low temperatures, both pristine and recovered hydrogel solutions, behaved as a viscous liquid (G″>G′). Storage and loss moduli increased with temperature due to enhanced interpolymer interaction. The gelation temperature (T_{gel}) is the crossing point of G′ and G″ (G₀=G″, tan d=1), which shows the starting point where the system changes from a viscous fluid into an elastic network.

A more elastic gel was formed as the temperature was raised, evidenced by the steady increase of storage modulus in FIG. 14B compared with loss modulus. A sharp increase of moduli occurred in both solutions at 25° C., with gel strength improved by more than two orders of magnitude, indicative of the coil to globule transition, and a second crossing point of G′ and G″ was also included in this region. The temperature ramp scans were compared of TRS64-10% stored at 10° C. and TRS64-10% in PUR stored at 50° C. for 48 hours followed by 10 minutes cooling at ice bath. The data show that the LCST of the TRS64-10% does not change. Also, the viscoelastic properties of heated hydrogel solution are effectively unchanged from the initial solution (FIG. 14B).

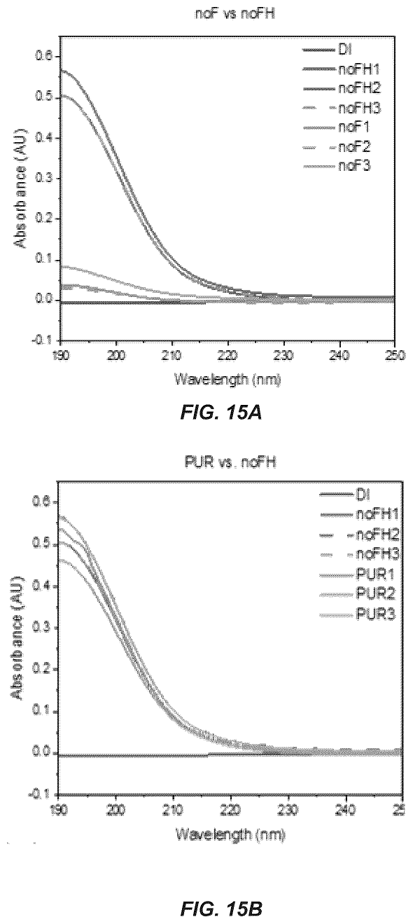

FIG. 15A shows a graph of absorbance versus wavelength of noFH samples compared with noF samples heated at approximately at 50° C. FIG. 15B shows a graph of absorbance versus wavelength of noFH samples compared with PUR samples heated at approximately at 50° C.

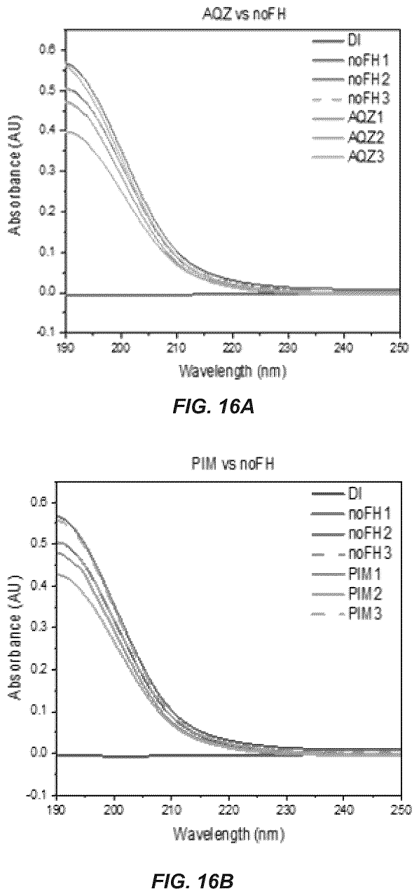

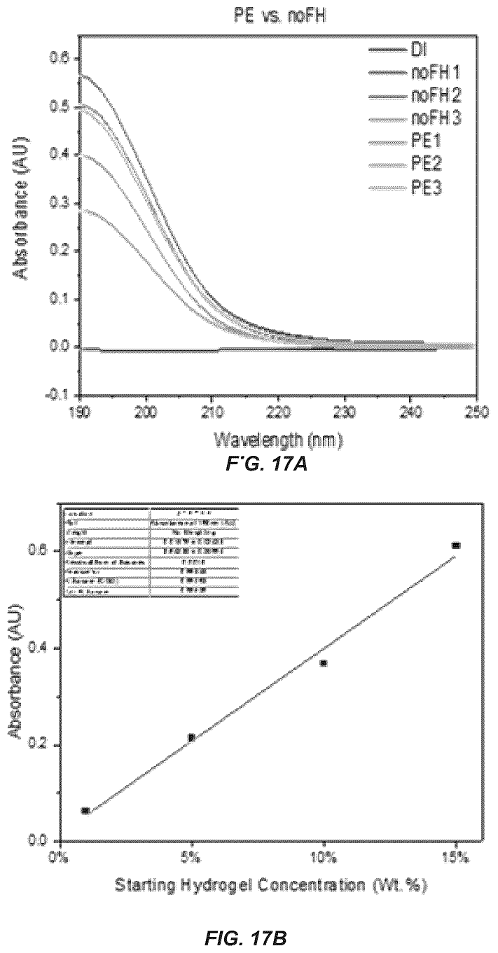

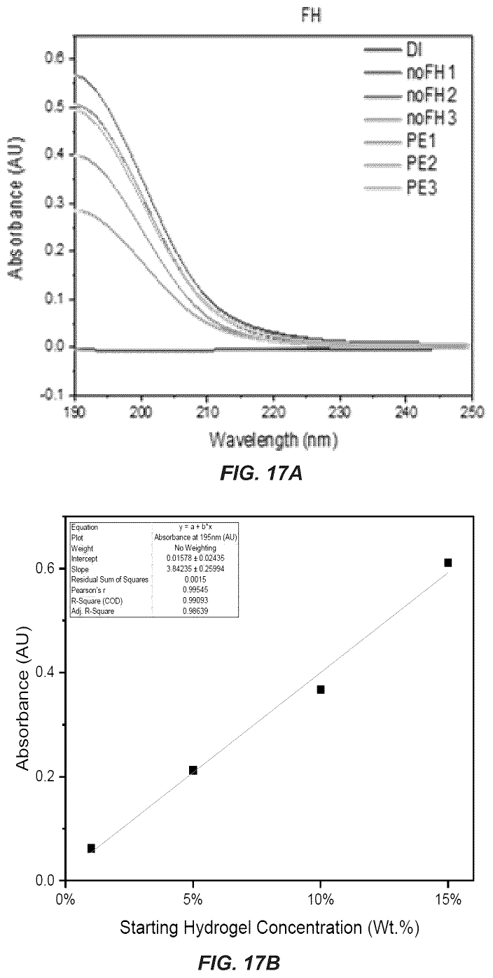

FIGS. 15A to 17B show graphs of absorbance versus wavelength of various examples with certain samples heated at 50° C. (noF) for 1 hour versus reference (noFH), reveals that warming the TRS64-10% retards the gel-sol transition progressed completely in 10 minutes cooling at water-ice bath. Specifically, FIG. 15A is a graph showing noFH samples compared with noF samples heated at approximately at 50° C. FIG. 15B shows a graph of absorbance versus wavelength of noFH samples compared with PUR samples heated at approximately at 50° C. FIG. 16A shows a graph of absorbance versus wavelength of noFH samples compared with AQZ foam samples heated at approximately at 50° C. FIG. 16B shows a graph of absorbance versus wavelength of noFH samples compared with PIM foam samples heated at approximately at 50° C. FIG. 17A shows a graph of absorbance versus wavelength of noFH samples compared with PE foam samples heated at approximately at 50° C. FIG. 17B shows a graph that includes a calibration curve (e.g., UV absorbance versus starting hydrogel concentration) to estimate the starting concentration of the recovered solution concentration based on their UV-Vis absorbance. The depicted graphs evidences enhanced recovery of the TRS by a factor of 8-10 when a host material is present in the syringe, even for the closed-cell PE foam.

Although systems and methods have been described with reference to specific embodiments, it will be understood by those skilled in the art that various changes may be made without departing from the spirit or scope of the disclosure. Accordingly, the disclosure of embodiments is intended to be illustrative of the scope of the disclosure and is not intended to be limiting. It is intended that the scope of the disclosure shall be limited only to the extent required by the appended claims. For example, to one of ordinary skill in the art, it will be readily apparent that any element of FIGS. 1-17B may be modified, and that the foregoing discussion of certain of these embodiments does not necessarily represent a complete description of all possible embodiments. For example, one or more of the procedures, processes, or activities of systems and methods of this disclosure may include different procedures, processes, and/or activities and be performed by some different operation in some different order.

All elements claimed in any particular claim are essential to the embodiment claimed in that particular claim. Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

Examples of this disclosure can be used as sealants in biomedical applications, such as ocular sealants as well as for wound closure. In some aspects, the herein disclosed enhanced hydrogel solution can be configured for on-demand deployment and be stored at ambient temperature (e.g., approximately ca. 25° C.) prior to use and then cooled before use. When cooled, copolymerization of the polymer (e.g., pNIPAM) in suspension returns to a homogeneous solution that can be applied to a target (e.g., as an ocular sealant, to a wound site, etc.). The multiple phase transitions involved in the storage and application of TRS solutions, such as PNB, pNIPAM, etc., can be completely reversible and rapid (e.g., less than 10 minutes) with no change in properties.

In some aspects, the herein disclosed enhanced hydrogel is formed with mechanical strength of higher molecular weight hydrogels and a fast reversible phase transition and demonstrated to include a fast reversible phase transition for higher molecular weight hydrogel that can be achieved if aspects of the hydrogel solution are stored within a high surface area host material. In some aspects, the herein disclosed enhanced hydrogel can be a homogenous hydrogel capable of recovering by using host material (e.g., polyurethane foam) after heating to 50° C. for 24 hours. Moreover, the gel-sol transition rate of the herein disclosed enhanced hydrogel (e.g., TRS64-10% with a host material, as described herein) is configured to increase at least eight to ten-fold relative to hydrogel heated without host material. The herein discussed example rheological analysis of the recovered solution after hosting the hydrogel within host material (e.g., PU foam) and storing at 50° C. demonstrates that storage in an open-cell foam host does not change the mechanical strength or LCST of the hydrogel. SEM images demonstrate the uniform morphology of the enhanced hydrogel when particles are stored within the pores of the host material. Accordingly, using a host material (e.g., foam) with a temperature-responsive hydrogel is demonstrated to ensure rapid and reversible gel-sol phase transitions in applications where the hydrogel can be exposed to high temperatures (e.g., during storage).

The specific configurations, choice of materials, concentrations thereof, steps in preparing, and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

What is claimed is:

1. A method for enhancing a gel to sol transition of a polymer comprising:
disposing the polymer on a host material;
exposing the polymer and the host material to an exposure temperature that causes the polymer to form a gel; and
cooling the polymer and the host material to a cooling temperature that causes the gel to transition to a sol.

2. The method of claim 1, further comprising:
upon cooling the polymer and the host material to the cooling temperature for a cooling duration of time, the gel becomes homogenous with one or more particles of the polymer stored within pores of the host material, the homogenous temperature-responsive hydrogel comprising a storage and loss moduli that increases with temperature due to enhanced interpolymer interaction.

3. The method of claim 2, wherein the cooling duration of time is between about 5 and 15 minutes.

4. The method of claim 1, wherein the exposing the polymer and the host material to the exposure temperature is for an exposure duration of time of at least 1 hour.

5. The method of claim 1, wherein the exposing the polymer and the host material to the exposure temperature is for an exposure duration of time of at least 24 hours.

6. The method of claim 1, wherein the exposure temperature is from about 25 to 75° C.

7. The method of claim 1, wherein the cooling temperature is less than about 25° C.

8. The method of claim 1, wherein the polymer is poly(N-isopropylacrylamide-co-butyl acrylate) (pNIPAM).

9. The method of claim 1, wherein a molecular weight of the polymer is at least one of Mₙ=64 kg mol⁻¹, Mₙ=41 kg mol⁻¹, Mₙ=35 kg mol⁻¹, Mₙ=24 kg mol⁻¹, and Mₙ=7 kg mol⁻¹.

10. The method of claim 1, wherein the host material comprises an open-cell foam, wherein the open-cell foam is selected from the group consisting of polyurethane (PUR), polyimide (PIM), hydrophilic polyurethane (PUR), Aquazone® (AQZ), and neoprene (NEO).

11. The method of claim 1, wherein a sol recovery rate resulting from the step of cooling is from about 74 to 99 percent.

12. The method of claim 1, wherein the host material comprises polyurethane (PUR) foam, and wherein a sol recovery rate resulting from the step of cooling is approximately 100%.

13. The method of claim 1, wherein a sol recovery rate resulting from the step of cooling is improved by a factor of about 8-10 relative to the polymer alone without the host material.

14. The method of claim 1, wherein pores of the host material range between about 0.2-1.1 mm.

15. A method for reversibly sealing tissue damage, comprising:
applying a temperature-responsive hydrogel to a tear or perforation in a tissue of a subject in an amount effective to seal the tear, wherein when exposed to a temperature above its critical solution temperature, the temperature-responsive hydrogel becomes adhesive, and when exposed to a temperature below its critical solution temperature, the temperature-responsive hydrogel becomes less adhesive wherein the temperature-responsive hydrogel comprises a polymer disposed on a host material.

16. The method of claim 15, further comprising:
prior to the step of applying and upon cooling the polymer and the host material to a cooling temperature for a cooling duration of time, the temperature-responsive hydrogel becomes homogenous with one or more particles of the polymer stored within pores of the host material, the homogenous temperature-responsive hydrogel comprising a storage and loss moduli that increases with temperature due to enhanced interpolymer interaction.

17. The method of claim 16, wherein the cooling duration of time is between about 5 and 15 minutes.

18. The method of claim 15, wherein the polymer is poly(N-isopropylacrylamide-co-butyl acrylate) (pNIPAM).

19. The method of claim 15, wherein the polymer is poly(N-isopropylacrylamide-co-N-tert-butylacrylamide) and poly(N-isopropylacrylamide), Poly(N,N-diethyl acrylamide), and poly(methyl vinyl ether), poly(N-vinyl caprolactam), oly(ethylene glycol), poly(propylene glycol), poly(vinylalcohol), poly(N-isopropylacrylamide), poly(methyl vinyl ether), poly(N-vinyl caprolactam), copolymer of poly(ethylene oxide) and poly(propylene oxide) s, and elastin-like oligo- and polypeptides.

20. A gel forming composition, comprising:
an aqueous solution;
a host material; and
a polymer disposed in pores of the host material, wherein the host material and the polymer are suspended in the aqueous solution;
wherein upon exposing the polymer and host material to an exposure temperature that causes the polymer to form a temperature-responsive hydrogel gel and cooling the polymer and the host material to a cooling temperature that causes the temperature-responsive hydrogel gel to transition to a sol.
